(19) United States
(12) Patent Application Publication
(10) Pub. No.: US 2009/0036548 A1
(43) Pub. Date: Feb. 5, 2009

(54) Mascara and liquid eyeliner enhancer

(76) Inventors: Julie Wardleigh, Spraks, NV (US); Theresa Kawchack, Wallawalla, WA (US)

Correspondence Address: Mark Levy;HINMAN, HOWARD & KATTELL, LLP, 80 Exchange Street, P.O. Box 5250, BINGHAMTON, NY, 13901, US

(21) Appl. No.: 11/881,865

(22) Filed: Jul. 30, 2007

(51) Int. Cl. A61K 47/44; A61K 47/04; A61K 47/12

(52) U.S. Cl. 514/769; 514/772; 514/784

(57) ABSTRACT

A composition and method of use of a composition for enhancing the usability of eye cosmetics, including but not limited to mascara and liquid eyeliner. The composition and its formulation contains a liquid enhancer, such that a few drops of the liquid enhancer added to the mascara and/or liquid eyeliner are used to maintain the viscosity, consistency, and smoothness of the cosmetic at the levels of the original, newly provided cosmetic. This enhancement thus allows continued ease of use and longer useful life of the cosmetic material.

### FIELD OF THE INVENTION

This invention pertains to mascara and liquid eyeliners and, more particularly, to a composition for enhancing cosmetics such as but not limited to mascara and eyeliner, the composition comprising a mixture of oils, stabilizers, and preservatives, in a liquid carrier, said preservatives comprising both oleophilic and hydrophilic materials, and said stabilizers comprising metal ion sequestrants preserving the solubility of the composition.

The composition and its formulation constitutes a liquid enhancer, such that a few drops of the liquid enhancer added to the mascara and/or liquid eyeliner will maintain the viscosity, consistency, and smoothness of the cosmetic, at the levels of the original, newly provided cosmetic. This enhancement thus allows continued ease of use and longer useful life of the cosmetic material.

### BACKGROUND OF THE INVENTION

Cosmetics are generally used for coloration and to make more noticeable eyelashes and the perimeter of the eyes. Various cosmetics, in the form of liquids, solid cakes, and thixotropic creams, are used to make eyelashes darker, make them appear longer and thicker, and in general to emphasize their appearance, and to make them more noticeable. In addition to cosmetic materials in liquid form, such materials are also available in the form of pencils of varying degrees of hardness, from hard powder based formulations to soft wax based formulations, which are used to outline the perimeter of the eyes and the eyelids. These cosmetics are referred to as eye liners and are used to emphasize the appearance of the eye by providing a pronounced and readily visible border to the eyes, in colors that may contrast or harmonize with skin tone and eye color. The applied eyeliner may simply outline the eyelids, or it may extend and enhance the shape of the eyelids and the eyes.

In each case, the successful application of the liquid and cream form of these cosmetics to the sensitive area surrounding the eyes requires a smooth, clump-free consistency of a uniform thickness and viscosity. These characteristics are typical of these cosmetics when they are newly provided to the user.

The useful life of the liquid and cream form of these cosmetics is naturally limited by their tendency to dry out. As the volatile constituents of these cosmetics are compromised, they lose smoothness by clumping and becoming more viscous, developing a stiffer consistency, thus becoming more difficult and eventually impossible to apply in the manner that was appropriate when they were newly opened and used.

Prior methods of formulating mascara and eyeliner and related cosmetics include the following referenced patents.

U.S. Pat. No. 7,090,860 to Yousfi et al. for TOPICAL COMPOSITION shows a cosmetic composition in stick form that includes the possible use of organic oils, water, hydrophilic additives, and mixtures thereof.

U.S. Pat. No. 7,030,985 to Jager-Lezer et al. for COLORED TRANSPARENT OR TRANSLUCENT COSMETIC COMPOSITION shows a cosmetic composition that may be used as a lipstick, and that also includes the possible use of organic oils, as well as aqueous gels and water soluble dyes.

U.S. Pat. No. 6,946,123 to De La Poterie et al. for FILM-FORMING COSMETIC COMPOSITION shows a cosmetic composition for mascara that is removable with hot water. The cosmetic composition includes the possible use of aqueous dispersions of oil soluble additives, including the use of vegetable oils.

U.S. Pat. No. 6,967,024 to Scancarella et al. for LONG WEARING COMPOSITION FOR MAKING UP EYES, SKIN, AND LIPS shows a cosmetic composition for use as mascara, eyeliner, or lip coating, in liquid form, whose composition includes the possible use of organic liquids.

U.S. Pat. No. 6,126,929 to Mougin for COSMETIC COMPOSITION COMPRISING A MIXTURE OF POLYMER PARTICLES CAPABLE OF BEING FILM-FORMED AND PARTICLES NOT CAPABLE OF BEING FILM-FORMED shows a cosmetic composition for use as a keratin fiber coating consisting of an aqueous dispersion whose composition includes the possible use of an oil.

Each of the referenced patents shows the possible use of one or more of the following ingredients: oils, organic oils, oil soluble materials, water, aqueous dispersions, and water soluble materials. None of the referenced patents, however, shows the use of a formulation to enhance the usability of a cosmetic by maintaining a suitable lower viscosity, maintaining smoothness and consistency, and preventing clumping.

### SUMMARY OF THE INVENTION

In accordance with the present invention, there is provided a composition of materials to be added to mascara or eyeliner cosmetics during the useful life of these cosmetics. This composition comprises a physiologically acceptable mixture of materials that is compatible with the original cosmetics and is stable over time. The inventive composition includes a number of constituents in an aqueous mixture including organic oils, stabilizers that prevent bacterial growth, stabilizers that prevent deleterious oxidation of organic materials, and stabilizers to prevent precipitation of solids from ionic materials in the mixture. The addition of a few drops of this composition enables the maintenance of the viscosity, consistency, and smoothness of the cosmetic at the levels of the original, new cosmetic, and thus provides for the continued ease of use and longer useful life of the cosmetic material. These enhancements are provided by the inventive composition in the following four ways:

1) the inventive composition achieves long term stability by enhancing viscosity, smoothness, and ease of application by compensating for the loss of volatile constituents, both volatile oils and water;

2) the inventive composition achieves long term stability by preventing the formation of insoluble precipitates that may affect viscosity, smoothness and clumping;

3) the inventive composition achieves long term stability by preventing the oxidation of organic oil constituents of the enhancing composition and of the cosmetics to be enhanced; and

4) the inventive composition achieves long term stability by preventing the growth of micro-organisms in the enhancing composition and in the cosmetics to which the composition may be added.

### DETAILED DESCRIPTION OF THE PREFERRED EMBODIMENT

In the use of cosmetics, and particularly the use of liquid and cream forms of mascara and eyeliner, the viscosity, consistency, and smoothness of the cosmetics is important to enhance their ease of use and their ability to be precisely and effectively applied. Over time, the cosmetics are subject to the loss of volatile constituents which can deleteriously affect these important properties.

In order to replace these volatile constituents, a physiologically acceptable mixture of materials is required that is compatible with the original cosmetics and is stable over time. To that end, an aqueous mixture is required including organic oils, preservers that prevent bacterial growth, preservers that prevent deleterious oxidation of organic materials, and stabilizers to prevent precipitation of solids from ionic materials in the mixture.

A variety of organic oils can be used, such as soybean oil, corn oil, wheat germ oil, and lemon oil, among others known to those skilled in the art. These oils can be preserved with regard to oxidation by oil soluble (oelophilic) antioxidants including d-alpha tocopherol acetate, butylated hydroxytoluene, and ascorbyl palmitate, among others. The commonly used water soluble antimicrobial materials such as sodium borate, boric acid, and sorbic acid, are physiologically compatible and can be used to preserve hydrophilic constituents of the mixture.

Finally, sequestrants such as glucono delta-lactone, sodium gluconate, potassium gluconate, and disodium ethylene diamine tetra-acetate can be used to prevent the precipitation of inorganic ions that may be present in the mixture, or encountered in use.

The constituents described, when added in appropriate proportion, can be mixed into a stable aqueous dispersion, and used to enhance the life and usability of mascara and/or eyeliner, by the addition of a few drops of the enhancing liquid mixture to the cosmetics, as necessary, to maintain viscosity, smoothness, and consistency of the cosmetic, at the levels of the original, newly provided cosmetic.

The aqueous solution, or liquid carrier, would typically comprise sterile de-ionized water and sodium chloride. The liquid carriers are used in an amount ranging from 80% to 95% by weight relative to the total weight of the liquid enhancer. The oils are present in an amount ranging from 1.0% to 16% by weight relative to the total weight. The hydrophilic preservatives are present in an amount ranging from 0.1% to 1.1% by weight, the oleophilic preservatives in amounts ranging from 1% to 3% by weight and the stabilizers in amounts ranging from 0.2% to 0.4% by weight, each weight relative to the total weight of the liquid enhancer.

The constituents can be used in various proportions; the composition with the preferred embodiment of this liquid enhancer is shown below.

### Composition:

### Percent Composition by Weight:

| | Range | Preferred |
|---|---|---|
| Sodium Chloride | 0.77-0.92% | 0.9% |
| Boric Acid | 0.43-0.51% | 0.5% |
| EDTA Disodium | 0.26-0.31% | 0.3% |
| Sodium Borate | 0.17-0.21% | 0.2% |
| Sorbic Acid | 0.17-0.21% | 0.2% |
| Vitamin E (d-alpha | 1.3-10.7% | 2.0% |
| tocopherol acetate) | | |
| Soybean Oil | 0.2-3.2% | 0.6% |
| Corn Oil | 0.2-3.2% | 0.6% |
| Wheat Germ Oil | 0.2-3.2% | 0.6% |
| Sesame Oil | 0.2-3.2% | 0.6% |
| Lemon Oil | 0.2-3.2% | 0.6% |
| Sterile De-ionized | 80-95% | |
| Water | | |

### Formulation by Weight, Used to Make 1 kg:

| | Range | Preferred |
|---|---|---|
| Sodium Chloride | 7.7-9.2 gm | 9.0 gm |
| Boric Acid | 4.3-5.1 gm | 5.0 gm |
| EDTA Disodium | 2.6-3.1 gm | 3.0 gm |
| Sodium Borate | 1.7-2.1 gm | 2.0 gm |
| Sorbic Acid | 1.7-2.1 gm | 2.0 gm |
| Vitamin E (d-alpha | 13.0-107.0 gm | 20.0 gm |
| tocopherol acetate) | | |
| Soybean Oil | 2.0-32.0 gm | 6.0 gm |
| Corn Oil | 2.0-32.0 gm | 6.0 gm |
| Wheat Germ Oil | 2.0-32.0 gm | 6.0 gm |
| Sesame Oil | 2.0-32.0 gm | 6.0 gm |
| Lemon Oil | 2.0-32.0 gm | 6.0 gm |

Sterile De-ionized Water sufficient to make 1000 gm approximately 950 gm

This technology can also be used to treat cosmetics in anticipation of future events, for example, before they might become less useable as their viscosity changes by loss of volatile constituents, or when it is desired to reduce their viscosity and make them of a more liquid consistency, for ease of application.

Since other modifications and changes varied to fit particular operating requirements and environments will be apparent to those skilled in the art, the invention is not considered limited to the examples chosen for purposes of disclosure and covers all changes and modifications which do not constitute departures from the true spirit and scope of the invention.

Having thus described the invention, what is desired to be protected by Letters Patent is presented in the subsequently appended claims.

What is claimed is:

1. A composition for enhancing cosmetics, the composition comprising a mixture of organic oils, stabilizers, and preservatives, in a liquid carrier, said preservatives comprising both oleophilic and hydrophilic materials, and said stabilizers comprising sequestrants preserving the solubility of the composition.

2. The composition for enhancing cosmetics in accordance with claim 1, wherein said liquid carrier comprises a sterile, isotonic solution of sodium chloride in de-ionized water.

3. The composition for enhancing cosmetics in accordance with claim 1, wherein said organic oils are selected from the group of: soybean oil, corn oil, wheat germ oil, sesame oil, and lemon oil.

4. The composition for enhancing cosmetics in accordance with claim 1, wherein said hydrophilic preservatives are selected from the group of: sodium borate, boric acid, and sorbic acid.

5. The composition for enhancing cosmetics in accordance with claim 1, wherein said oleophilic preservatives are selected from the group of: d-alpha tocopherol acetate, butylated hydroxytoluene, and ascorbyl palmitate.

6. The composition for enhancing cosmetics in accordance with claim 1, wherein said metal ion sequestrants are selected from the group of: glucono delta-lactone, sodium gluconate, potassium gluconate, and disodium ethylene diamine tetra-acetate.

7. The composition for enhancing cosmetics in accordance with claim 1, wherein said liquid carrier is present in an amount ranging from about 80% to 95% by weight relative to the total weight of said composition.

8. The composition for enhancing cosmetics in accordance with claim 1, wherein said organic oils are present in an amount ranging from about 1% to 16% by weight relative to the total weight of said composition.

9. The composition for enhancing cosmetics in accordance with claim 1, wherein said hydrophilic preservatives are present in an amount ranging from about 0.1% to 1.1% by weight relative to the total weight of said composition.

10. The composition for enhancing cosmetics in accordance with claim 1, wherein said oleophilic preservatives are present in an amount ranging from about 1% to 3% by weight relative to the total weight of said composition.

11. The composition for enhancing cosmetics in accordance with claim 1, wherein said stabilizers are present in an amount ranging from about 0.2% to 0.4% by weight relative to the total weight of said composition.

12. A method of enhancing the usability of a cosmetic, such as, but not limited to, mascara and eyeliner by applying an enhancing liquid comprising, in a physiologically acceptable medium comprising an aqueous phase: a mixture of organic oils, hydrophilic and oleophilic preservatives, and sequestrants, to said cosmetics for replacing lost volatile constituents of said cosmetics and maintaining viscosity, consistency, and smoothness thereof at the levels of the original, newly provided cosmetics.

13. A liquid cosmetic enhancer, comprising:
an aqueous solution;
an oleophilic preservative;
a hydrophilic preservative;
a mixture of oils; and
a stabilizing sequestrant;
whereby addition of the liquid cosmetic enhancer to a liquid cosmetic enhances the viscosity, consistency and smoothness thereof.

14. The liquid cosmetic enhancer in accordance with claim 13, wherein the mixture of oils comprises at least two different oils.

15. The liquid cosmetic enhancer in accordance with claim 14, wherein the mixture of oils comprises at least one of a soybean oil, a corn oil, a wheat germ oil, a sesame oil, and a lemon oil.

16. The liquid cosmetic enhancer in accordance with claim 15, wherein said soybean oil, corn oil, wheat germ oil, sesame oil, and lemon oil are combined in the mixture in approximately equal amounts.
